(19) United States
(12) Patent Application Publication
(10) Pub. No.: US 2007/0156079 A1
(43) Pub. Date: Jul. 5, 2007

(54) Glaucoma Treatment Devices and Methods

(75) Inventor: J. David Brown, St. Paul, MN (US)

Correspondence Address: FISH & RICHARDSON P.C., PO BOX 1022, MINNEAPOLIS, MN, 55440-1022, US

(73) Assignee: BG Implant, Inc., St. Paul, MN (US)

(21) Appl. No.: 11/531,965

(22) Filed: Sep. 14, 2006

Related U.S. Application Data
(60) Provisional application No. 60/717,592, filed on Sep. 16, 2005.

(51) Int. Cl. A61M 5/00

(52) U.S. Cl. 604/9

(57) ABSTRACT

This document provides methods and materials related to treating glaucoma. For example, devices that can be implanted into a human's eye to treat glaucoma, methods for treating glaucoma, compositions for reducing polypeptide clogging of implanted devices, and methods for making devices for treating glaucoma are provided.

### CROSS-REFERENCE TO RELATED APPLICATIONS

This application claims the benefit of U.S. Provisional Application Ser. No. 60/717,592, filed Sep. 16, 2005.

### BACKGROUND

1. Technical Field

This document provides devices and methods related to treating glaucoma.

2. Background Information

Glaucoma is the leading cause of irreversible blindness in the world. It is estimated that 70 million people worldwide have glaucoma, and that nearly 7 million are bilaterally blind from this disease. In the United States, 2.5 to 3 million people suffer from glaucoma, and it is the third most common reason for adults to visit a medical doctor. Elevated intraocular pressure is the outstanding risk factor for the development of glaucoma, and the main reason for progression of the disease. Accordingly, treatment of glaucoma has been focused on lowering the intraocular pressure in the affected eye.

Glaucoma treatment has customarily comprised a three-step process. First, medicines are tried, such as beta-adrenergic antagonists, alpha-adrenergic agonists, carbonic anhydrase inhibitors, and prostaglandin analogues. These have proven only moderately, and inconsistently, effective, and can lead to many, sometimes life threatening, side effects, such as allergic, respiratory, and cardiac side-effects. If medical treatment is either not effective or not tolerated, laser trabeculoplasty (LT) is usually the next step. LT success is often limited, and is ultimately temporary. The final therapeutic step involves surgery. Trabeculectomy is by far the most common type of surgery done for treatment of glaucoma. It was first described by Cairns in 1969, slightly modified by Watson 1969-71, and has changed little during the last three decades. In a trabeculectomy, a hole is made in the eye near the limbus and into the anterior chamber, under an overlying scleral flap. The aqueous humor thereby is allowed to drain into the subconjunctival space. Subsequent scarring circumscribes this area of subconjunctival drainage into a bleb. Sometimes, the scarring progresses to completely scar down the bleb, stopping the flow of aqueous humor, and causing the surgery to fail. Mitomycin C, an anti-fibroblastic drug, has been used to combat scarring attendant to trabeculectomy. While increasing surgical success, however, the use of this drug has significantly added to the risks and complications of filtering surgery; mitomycin C causes thinning of the conjunctiva and can lead to leaking through the thinned conjunctiva, and such leaking often leads to hypotony and intraocular infection.

Glaucoma drainage devices (GDD) are an attempt to control the scarring which so commonly tends to seal conduits made in tissue. Molteno, in 1969, described the first of the currently used type of GDD. They consist of a tube and a plate made of synthetic biomaterials. The tube is inserted into the anterior chamber and conducts the aqueous humor to the plate, which is in the subconjunctival space. The problem remains, however, of scarring of the bleb which forms around the plate. About 80% of GDDs appear to be successful for one year, with a 10% additional failure rate each year thereafter. There are significant complications associated with these devices, both in the perioperative and postoperative periods, including hypotony, flat anterior chamber, suprachoroidal hemorrhage, retinal detachment, a hypertensive phase, endophthalmitis, diplopia, corneal decompensation, conjunctival melting, and others. One or more complications have been found to occur in 60-70% of cases.

### SUMMARY

This document provides methods and materials related to treating glaucoma. For example, this document provides devices that can be implanted into a human's eye to treat glaucoma. In some cases, such devices can contain a flexible filter capable of providing outflow resistance to aqueous humor flowing through a lumen of the device and capable of flexing in response to an increase in intraocular pressure. Such flexing can allow the outflow resistance of aqueous humor to change as the intraocular pressure changes. For example, the resistance to aqueous humor outflow can be reduced as intraocular pressure increases. Devices having a flexible filter can provide patients with a device that can normalize intraocular pressure over time, thereby providing pressure homeostasis.

This document also provides methods and materials for making devices to treat glaucoma. For example, this document provides methods and materials for using heat shrinkable materials to form a device having a lumen and filter. Such devices can be one-piece products and can be conveniently produced in a uniform manner.

In addition, this document provides methods and materials that can be used to reduce protein/polypeptide clogging of devices implanted into an eye. For example, this document provides eye drop solutions having biodegradable particles coated with one or more proteases (e.g., papain) capable of cleaving polypeptides, coated with one or more surfactants capable of disrupting hydrophobic interactions (e.g., Triton X-100), or coated with a combination thereof. Such solutions can allow patients to self-administer a composition that helps maintain the effectiveness of an implanted device.

This document also provides methods and materials for determining or monitoring intraocular pressure. For example, this document provides detectors that can emit light into an eye containing an implanted device and can detect the wavelength of reflected light. The implant can be designed to contain a flexible filter having a pressure sensor that reflects light at a particular wavelength depending upon the degree of filter flexing caused by intraocular pressure. For example, an un-flexed filter can reflect light at a particular wavelength, which can indicate low or normal intraocular pressure, while a fully flexed filter can reflect light at a different wavelength, which can indicate substantially elevated intraocular pressure. Having the ability to measure intraocular pressure can provide clinicians with the ability to assess the effectiveness of an implanted device as well as the state of a patient's glaucoma.

In general, one aspect of this document features a device for treating glaucoma in an eye, comprising, or consisting essentially of: (a) a body defining a lumen and having first and second ends and external and lumenal surfaces, the body having a length sufficient to provide fluid communication between the anterior chamber and tear film of an eye through the lumen when the device is implanted in the sclera; and (b) a flexible filter membrane capable of providing outflow resistance to aqueous humor flowing through the lumen and capable of flexing in response to an increase in intraocular pressure. The second end of the device can be adapted to lie substantially flush with the scleral surface when the device is implanted in the sclera. The body can be flared at the second end. The body can comprise a material selected from the group consisting of silicone, acrylic, polyimide, polypropylene, polymethyl methacrylate, polytetrafluoroethylene, hydrogels, polyolefin, polyvinylchloride, and polyester. The flexible filter membrane can comprise polydimethylsiloxane, a silicone rubber, or other polymers such as silastic or gel materials (e.g., a hydrogel). The flexible filter membrane can be a microporous/nanoporous filter membrane or a debris filter. The flexible filter membrane can be a microporous/nanoporous filter membrane and can comprise micropores having a diameter less than or equal to about 0.2 microns. The flexible filter membrane can be a debris filter and can comprise pores having a diameter between about 0.5 and 2 microns. The debris filter can comprise an inflow face, an outflow face, and a peripheral edge contiguous with the body. The device can comprise a microporous/nanoporous filter membrane and a debris filter. The debris filter can be positioned at the first end or between the first end and the microporous/nanoporous filter membrane. The flexible filter membrane can be positioned between the debris filter and the microporous/nanoporous filter membrane.

The microporous/nanoporous filter membrane can comprise a pressure sensor. The pressure sensor can comprises photonic crystals. The photonic crystals can be within a polymer network of a hydrogel. The body and the microporous/nanoporous filter membrane can comprise the same material. The body and the microporous/nanoporous filter membrane can be fused or bonded together using heat. The body and the microporous/nanoporous filter membrane can comprise polyolefin, polypropylene, polytetrafluoroethylene, polyvinylchloride, polyester, or another polymer. The device can comprise a second debris filter. The second debris filter can be positioned at or near the second end of the body, external to the microporous/nanoporous filter membrane. The flexing of the flexible filter membrane in response to an increase in intraocular pressure can reduce the outflow resistance.

The flexible filter membrane can comprise a pressure sensor. The pressure sensor can comprise photonic crystals. The photonic crystals are within a polymer network of a hydrogel. The body and the flexible filter membrane can comprise the same or different materials. The body and the flexible filter membrane can be fused or bonded together using heat. The body and the flexible filter membrane can comprise polyolefin, polypropylene, polytetrafluoroethylene, polyvinylchloride, polyester, or another polymer.

In another aspect, this document features a method for treating glaucoma, comprising, or consisting essentially of: (a) providing a device comprising a body defining a lumen and having first and second ends, the body having sufficient length to provide fluid communication between the anterior chamber and tear film of an eye, and the device comprising a flexible filter membrane capable of providing outflow resistance to aqueous humor and capable of flexing in response to an increase in intraocular pressure; and (b) implanting the device in the sclera of the eye such that aqueous humor flows from the anterior chamber to the tear film of the eye. The device can contain any of the features or configurations provided herein. For example, as described above, the flexible filter of the device can be a microporous/nanoporous filter membrane comprising a pressure sensor.

In another aspect, this document features a method for making a device for treating glaucoma in an eye. The method comprises, or consists essentially of, using heat to fuse or bond a body to a filter membrane to form the device, wherein the body comprises a lumen, first and second ends, and external and lumenal surfaces, the body having a length sufficient to provide fluid communication between the anterior chamber and tear film of an eye through the lumen when the device is implanted in the sclera, and wherein the filter membrane is capable of providing outflow resistance to aqueous humor flowing through the lumen. The device can contain any of the features or configurations provided herein. For example, as described above, the flexible filter of the device can be a microporous/nanoporous filter membrane comprising a pressure sensor. In addition, the body and the filter membrane can comprise the same or different materials. The body material can be a heat shrink material. The material can be selected from the group consisting of polyolefin, polypropylene, polytetrafluoroethylene, polyvinylchloride, polyester, and other polymers.

In another aspect, this document features a method for reducing clogging (e.g., polypeptide clogging) in a device implanted in the sclera of an eye. The method comprises, or consists essentially of, administering a solution comprising particles containing a protease, a surfactant, heparin, or a combination thereof to the eye under conditions wherein material (e.g., polypeptides) clogging the device are cleaved or removed. The device can contain any of the features or configurations provided herein. For example, as described above, the device can comprise a body defining a lumen and having first and second ends, the body having sufficient length to provide fluid communication between the anterior chamber and tear film of the eye, and the device comprising a filter membrane capable of providing outflow resistance to aqueous humor. The device can comprise a flexible filter membrane capable of flexing in response to an increase in intraocular pressure. The flexible filter membrane can be the filter membrane. The solution can be a biocompatible solution. The solution can be an eye drop solution. The particles can be capable of degrading following administration to the eye. The particles can comprise material selected from the group consisting of thermoplastic starch materials, mater-bi, polylatic acid, and poly-hydroxybutyrate-co-hydroxyvalerate. The protease can be a papain or subtilisin protease.

In another aspect, this document features a method for providing a patient with the ability to monitor intraocular pressure. The method comprises, or consists essentially of: (a) providing a patient with a detector comprising a light source and a wavelength sensor, wherein the sclera of an eye of the patient comprises (i) a device comprising a body defining a lumen and having first and second ends and external and lumenal surfaces, the body having a length sufficient to provide fluid communication between the anterior chamber and tear film of the eye through the lumen and (ii) a flexible filter membrane capable of providing outflow resistance to aqueous humor flowing through the lumen and capable of flexing in response to an increase in intraocular pressure, wherein the flexible filter membrane comprises a pressure sensor; and (b) instructing the patient to emit light from the detector onto the eye such that the detector is capable of detecting the wavelength of the emitted light that is reflected from the pressure sensor. The device can contain any of the features or configurations provided herein. For example, as described above, the flexible filter of the device can be a microporous/nanoporous filter membrane. The pressure sensor can comprise photonic crystals. The photonic crystals can be within a polymer network of a hydrogel of the flexible filter membrane. The light can be emitted as white light. The detector can record the wavelength of the emitted light that is reflected from the pressure sensor. The detector can convert the detected wavelength of the emitted light that is reflected from the pressure sensor into a pressure value. The detector can record the wavelength value of the emitted light that is reflected from the pressure sensor or a pressure value converted from the wavelength value, wherein the recorded wavelength value or pressure value is recorded with the time, day, or time and day that the detector detected the wavelength. The detector can record multiple wavelength values detected by the detector at different times or multiple pressure values converted from the multiple wavelength values.

In another aspect, this document features a method for determining intraocular pressure in a patient, wherein the sclera of an eye of the patient comprises, or consists essentially of: (i) a device comprising a body defining a lumen and having first and second ends and external and lumenal surfaces, the body having a length sufficient to provide fluid communication between the anterior chamber and tear film of the eye through the lumen and (ii) a flexible filter membrane capable of providing outflow resistance to aqueous humor flowing through the lumen and capable of flexing in response to an increase in intraocular pressure, wherein the flexible filter membrane comprises a pressure sensor. The method comprises, or consists essentially of: (a) providing a detector comprising a light source and a wavelength sensor; and (b) emitting light from the detector onto the eye of the patient such that the detector is capable of detecting the wavelength of the emitted light that is reflected from the pressure sensor. The device can contain any of the features or configurations provided herein. For example, as described above, the flexible filter of the device can be a microporous/nanoporous filter membrane. The pressure sensor can comprise photonic crystals. The photonic crystals can be within a polymer network of a hydrogel of the flexible filter membrane. The light can be emitted as white light. The detector can record the wavelength of the emitted light that is reflected from the pressure sensor. The detector can convert the detected wavelength of the emitted light that is reflected from the pressure sensor into a pressure value. The detector can record the wavelength value of the emitted light that is reflected from the pressure sensor or a pressure value converted from the wavelength value, wherein the recorded wavelength value or pressure value is recorded with the time, day, or time and day that the detector detected the wavelength. The detector can record multiple wavelength values detected by the detector at different times or multiple pressure values converted from the multiple wavelength values.

In another aspect, this document features a kit comprising, or consisting essentially of, a device and a detector, wherein the device comprises (a) a body defining a lumen and having first and second ends and external and lumenal surfaces, the body having a length sufficient to provide fluid communication between the anterior chamber and tear film of an eye through the lumen when the device is implanted in the sclera, and (b) a flexible filter membrane capable of providing outflow resistance to aqueous humor flowing through the lumen and capable of flexing in response to an increase in intraocular pressure, wherein the flexible filter membrane comprises a pressure sensor; and wherein the detector comprises a light source and a wavelength sensor, wherein the detector is capable of emitting light onto an eye containing the device such that the detector is capable of detecting the wavelength of the emitted light that is reflected from the pressure sensor. The device and detector can contain any of the features or configurations provided herein. For example, as described above, the flexible filter of the device can be a microporous/nanoporous filter membrane.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a mid-horizontal cross-sectional view of an eye with one embodiment of a device implanted and shown in longitudinal cross section.

FIG. 1B is an external view of an eye showing the external, intrascleral, and intra-anterior chamber portions of the device shown in FIG. 1A implanted in an eye.

FIG. 1C is an enlarged cross-sectional view of a flexible filter in an un-flexed position.

FIG. 1D is an enlarged cross-sectional view of a flexible filter in a flexed position.

FIG. 1E is an enlarged cross-sectional view of a flexible filter in an un-flexed position.

FIG. 1F is an enlarged cross-sectional view of a flexible filter in a flexed position.

FIG. 1G is an enlarged cross-sectional view of a portion of a flexible filter containing pressure sensors with the flexible filter in an un-flexed position.

FIG. 1H is an enlarged cross-sectional view of a portion of a flexible filter containing pressure sensors with the flexible filter in a flexed position.

FIG. 1I is an enlarged front view of a flexible filter containing pressure sensors with the flexible filter in a flexed position.

FIG. 2A is a mid-horizontal cross-sectional view of an eye with another embodiment of a device implanted and shown in longitudinal cross section.

FIG. 2B is an external view of an eye showing the external, intrascleral, and intra-anterior chamber portions of the device shown in FIG. 2A implanted in an eye.

FIG. 3A is a mid-horizontal cross-sectional view of an eye with another embodiment of a device implanted and shown in longitudinal cross section.

FIG. 3B is an external view of an eye showing the external, intrascleral, and intra-anterior chamber portions of the device shown in FIG. 3A implanted in an eye.

Like reference symbols in the various drawings indicate like elements.

### DETAILED DESCRIPTION

This document relates to methods and materials for treating glaucoma. In particular, this document relates to devices wherein a generally tubular body is provided which is of sufficient length to allow aqueous humor to flow from the anterior chamber of an afflicted eye through a lumen of the tubular body and into the tear film when the device is implanted in the sclera. A filter capable of providing outflow resistance to aqueous humor flowing through the lumen can be provided in the device. In some cases, the devices provided herein can contain a flexible filter that responds to pressure changes such that the outflow resistance decreases as intraocular pressure increases. The device may be implanted in the sclera of an afflicted eye to treat glaucoma.

The devices provided herein have numerous advantages. For example, the devices provided herein can drain aqueous humor into the tear film, rather than into the subconjuctival space. This can reduce the risk of developing, or prevent the development of, a conjunctival bleb, and therefore reduce or eliminate the potential to scar. In preferred embodiments, a filter portion can be fused or bonded to the body to form a one-piece device having a simple design and which can be easy and safe to insert into an afflicted eye. The filter can be readily accessible for vacuum, chemical, or enzymatic cleaning. Aqueous humor can be expelled into the tear film, enhancing moisture and lubrication in the eye. Also, in preferred embodiments, the filter can be comprised of a nanoporous/microporous membrane material. The nanoporous/microporous membrane can have pores sized to block all bacteria (e.g., less than 0.2 micron pore diameters), and pore number and length may be calculated to provide aqueous humor outflow that yields desirable intraocular pressure. The materials used to make the device can be selected to provide bulk biocompatibility by both seeking to match scleral rigidity, and by providing the portion of the device that is in contact with eye tissue with a porous cellular ingrowth surface to promote biointegration. Both the scleral rigidity compatibility and the biointegration can contribute to the elimination of micromotion of the device. The biointegration can also eliminate potential dead space around the device, thus reducing or removing the risk of a tunnel infection into the eye. The surfaces of the device can be coated with other materials, such as polymer coatings or biologically active molecules, to promote surface biocompatibility and/or immobilization of the implanted device. The devices provided herein can contain flexible filters so that the intraocular pressure within a patient's eye remains essentially constant even though aqueous humor flow fluctuates. In some cases, the devices provided herein can contain flexible filters having pressure sensors that allow intraocular pressure to be measured.

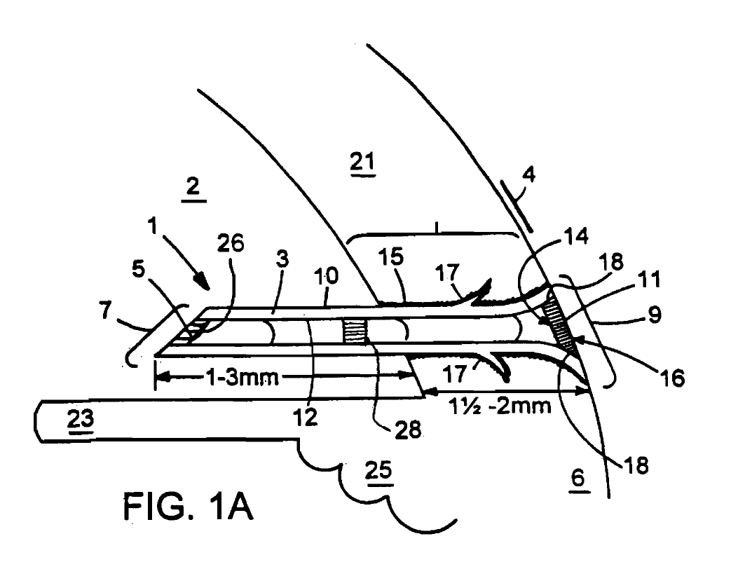

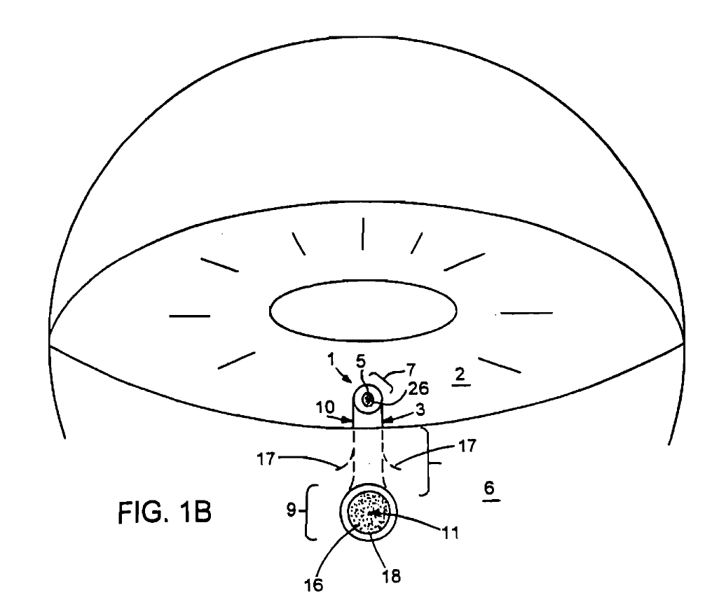

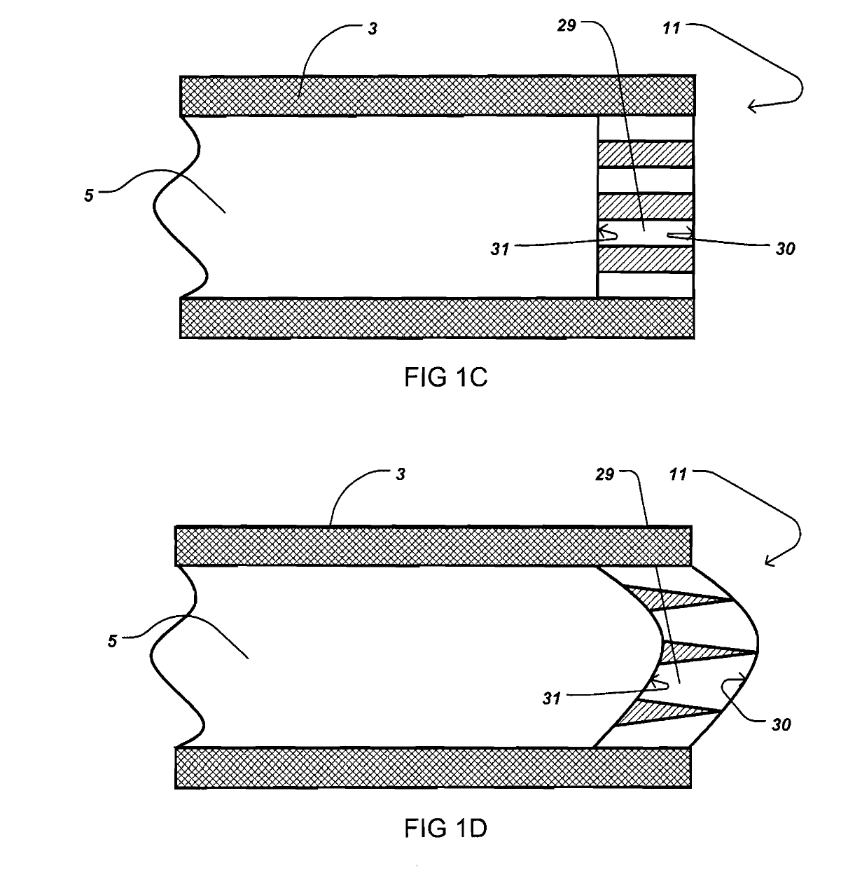

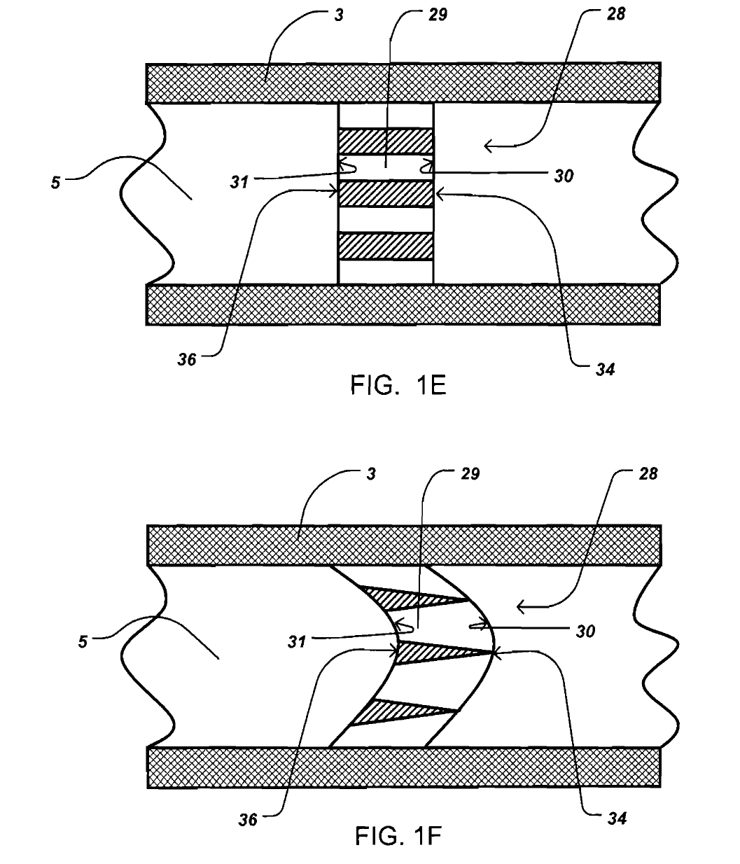

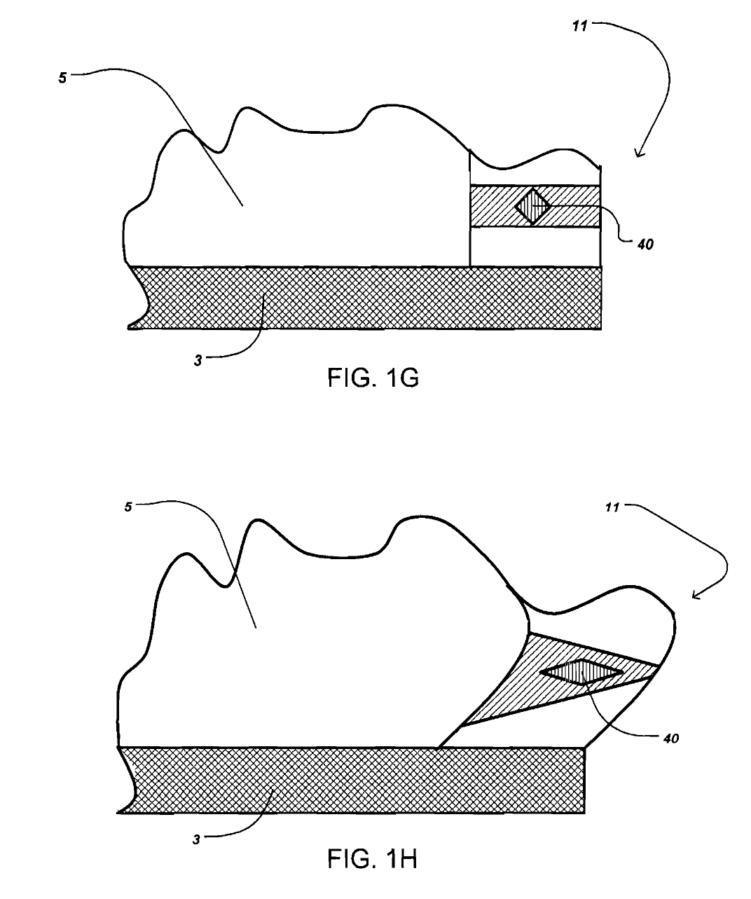

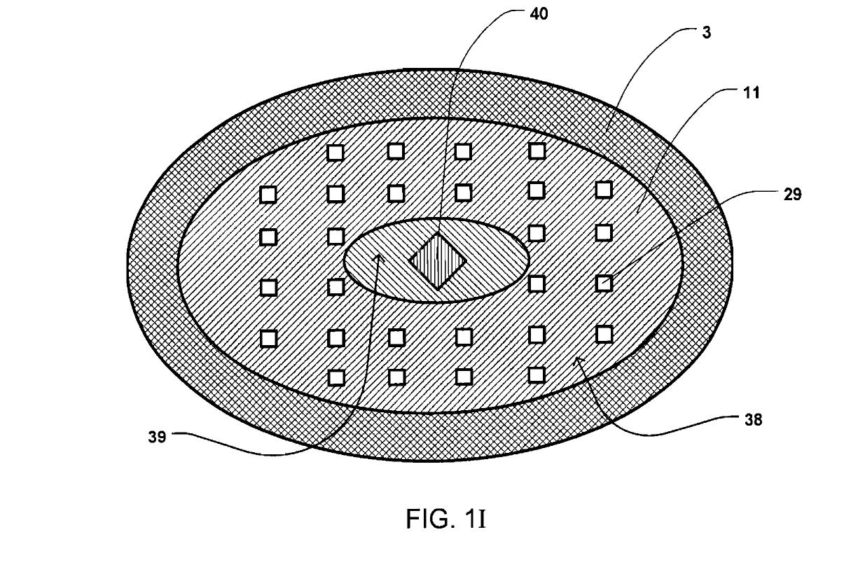

A device illustrative of one embodiment of this document is shown in FIGS. 1A and 1B. As shown in longitudinal cross-section in FIG. 1A as implanted in an eye, the device 1 can include a body 3 defining a lumen 5 and having a first end 7 and a second end 9. The body can have an external surface 10, and a lumenal surface 12. A filter 11 can be provided at the second end 9 of the device. The filter 11 can have an inflow face 14, and outflow face 16, and a peripheral edge 18. The device can have a length sufficient to provide fluid communication between the anterior chamber and tear film of an eye when the device is implanted in the sclera. The filter 11 can be capable of providing outflow resistance to aqueous humor flowing through the lumen 5. The device 1 can be implanted in the sclera 6 of the eye. Also shown in FIG. 1A are the cornea 21, the iris 23, and the ciliary body 25.

In some embodiments, filter 11 can be flexible. For example, filter 11 can be flexible such that an increase in intraocular pressure causes filter 11 to bow and increase the average diameter of its pores (e.g., the average of diameter measurements made at various points along a pore's length), thereby reducing the outflow resistance to aqueous humor flowing through the lumen 5. As shown in FIGS. 1C and 1D, filter 11 can be un-flexed in response to low or normal intraocular pressure (FIG. 1C), and flexed in response to increased intraocular pressure (FIG. 1D). A flexible filter can allow the device to maintain a stable intraocular pressure in spite of the fact that aqueous humor inflow can be variable over a day. Any flexible material can be used to make a flexible filter including, without limitation, polydimethylsiloxane, silicone rubbers, and hydrogel gels.

As described herein, the resistance of a filter (e.g., a microporous/nanoporous filter membrane) can be determined by two adjustable variables: the length of the pores (i.e., the membrane thickness) and the radius of the pores (i.e., it's radius to the fourth power). If the filter membrane is rigid, then the resistance remains constant under varying flow rates. For example, a rigid filter can be used to provide constant resistance during the day and night even though the inflow of aqueous humor is double during the day as compared to night. Because pressure equals resistance times flow, when the flow doubles, and the resistance stays the same, then the pressure also doubles. A flexible filter can be used as described herein so that the resistance can decrease in tandem with a flow increase, keeping the pressure relatively constant. In general, by making a porous filter membrane flexible, it will flex when the pressure increases inside the eye. This flexing can cause the pores to widen towards their external surface, while widening much less at their internal surface (FIGS. 1C and 1D). Thus, the pores can change from being generally cylindrical to non-cylindrical as the filter is flexed. In some cases, flexing a flexible filter can cause the inner pore radius to increase little, while causing the external radius to increase significantly. For example, as shown in FIG. 1C, the external pore diameter 30 of pore 29 can be similar to the internal pore diameter 31 of pore 29 when filter 11 is in an un-flexed position. When flexed, as shown in FIG. 1D, the external pore diameter 30 of pore 29 can be greater than the internal pore diameter 31 of pore 29.

In some cases, the smaller inner pore diameter can be designed to not widen to more than 0.2 microns. For example, in devices having a microporous/nanoporous filter membrane at the external surface of the device, bacterial ingress can be prevented by having a microporous/nanoporous filter membrane with pores where the inner pore diameter does not widen to more than 0.2 microns as the filter flexes. A device provided herein can lack such a flexible microporous/nanoporous filter membrane when, for example, a pressure responsive flexible filter membrane is included such as a flexible filter membrane located at a position other than the external surface of the device. For example, a device can have a rigid microporous/nanoporous filter membrane having pores with a minimum diameter that is capable of blocking bacterial ingress into the eye as well as a flexible filter membrane having pores with any length or diameter that provides, for example, pressure responsive resistance.

In addition, the shape of pore 29 can change from a generally cylindrical shape (FIG. 1C) to a non-cylindrical shape (FIG. 1D) as filter 11 flexes. A flexed, non-cylindrical pore can provide about 20 percent of the resistance of an un-flexed, cylindrical pore. A flexible porous membrane can be used to provide a homeostatic pressure control capable of compensating for flow variations. It would preferably be made out of a flexible polymer, using both bulk and surface micromachining.

Flexible filter 28 can be designed to provide the primary source of resistance to aqueous humor outflow. In addition, flexible filter 28 can be located anywhere along lumen 5 of the device. In some embodiment, instead of containing a flexible membrane, a device provided herein can contain a valve (e.g., a cantilever valve) or a flow resistor within the lumen to provide pressure responsive resistance to outflow. Such a valve or flow resistor can be designed to be self-adjusting by having outflow pores or channels that increase in size in response to an increase in pressure. In some cases, outflow resistance of the valve or flow resistor can be remotely adjusted using, for example, wireless technology such as an electromagnetic power source.

In some cases, a device provided herein can contain a flexible filter in addition to a rigid microporous/nanoporous filter membrane and a rigid debris filter. For example, with reference to FIG. 1A, device 1 can contain flexible filter 28. For example, flexible filter 28 can be flexible such that an increase in intraocular pressure causes filter 28 to bow and increase the average diameter of its pores (e.g., the average of diameter measurements made at various points along a pore's length), thereby reducing the outflow resistance to aqueous humor flowing through the lumen 5. As shown in FIGS. 1E and 1F, filter 28 can be un-flexed in response to low or normal intraocular pressure (FIG. 1E), and flexed in response to increased intraocular pressure (FIG. 1F). Again, a flexible filter can allow the device to maintain a stable intraocular pressure in spite of the fact that aqueous humor inflow can be variable over a day. With reference to FIGS. 1E and 1F, this flexing can cause the pores to widen towards their external surface (e.g., external surface 34), while widening much less at their internal surface (e.g., external surface 36). Thus, the pores can change from being generally cylindrical to non-cylindrical as the filter is flexed. In some cases, flexing a flexible filter can cause the inner pore radius to increase little, while causing the external radius to increase significantly. For example, as shown in FIG. 1E, the external pore diameter 30 of pore 29 can be similar to the internal pore diameter 31 of pore 29 when filter 28 is in an un-flexed position. When flexed, as shown in FIG. 1F, the external pore diameter 30 of pore 29 can be greater than the internal pore diameter 31 of pore 29. In addition, the shape of pore 29 can change from a generally cylindrical shape (FIG. 1E) to a non-cylindrical shape (FIG. 1F) as filter 28 flexes.

Any flexible material can be used to make a flexible filter including, without limitation, polydimethylsiloxane, silicone rubbers, and hydrogel gels. Filter 11 in devices containing flexible filter 28 can be primarily designed to prevent bacteria ingress. For example, devices containing a flexible filter (e.g., flexible filter 28) can contain a rigid microporous/nanoporous filter membrane that provides limited resistance to flow. Such a microporous/nanoporous filter membrane can contain an increased number of pores and/or can be thinner than a comparable microporous/nanoporous filter membrane designed to provide resistance to flow.

In some embodiments, the external most filter (e.g., filter 11 of FIG. 1A) can contain one or more pressure sensors. For example, with reference to FIGS. 1G and 1H, a flexible filter can contain one or more pressure sensors such as a crystalline colloidal array (CCA) of photonic crystals 40. The area of a filter/membrane containing a pressure sensor (e.g., the CCA) can be either porous or non-porous, and can comprise either the entire filter membrane or a smaller portion of the filter (e.g., a small inner circular region of a filter). For example, as shown in FIG. 11, flexible filter 11 can contain an outer ring 38 that contains pores (e.g., pore 29) and an internal disc 39 that is non-porous. Such a non-porous area can contain pressure sensors (e.g., photonic crystals represented as phonic crystal 40). In some cases, both outer ring 38 and internal disc 39 can contain pressure sensors. While shown as being disc and ring shaped, the porous and non-porous areas can be any shape including oval, square, or rectangular. In some cases, the entire filter is porous and contains pressure sensors.

Pressure sensors such as the CCA can change shape and other structural characteristics (e.g., density) as the flexible filter flexes. For example, photonic crystals 40 can have one shape and density when the flexible filter is un-flexed (FIG. 1G), and another shape and density when the flexible filter flexes (FIG. 1H). These different shapes and structural characteristics can allow the degree of flexing, and thus the amount of intraocular pressure, to be determined by virtue of the fact that reflected light can have a particular wavelength depending on the shape and structural characteristics of the CCA within the flexible filter. Examples of pressure sensors include, without limitation, photonic crystals in crystalline colloidal arrays such as those described elsewhere (Alexeev et al., Anal. Chem., 75:2316-23 (2003) and Alexeev et al., Clin. Chem., 12:2353-60 (2004)). Such CCAs can be embedded within, for example, a polymer network of a hydrogel (e.g., a polymer network of a polyacrylamide-poly (ethylene glycol) hydrogel).

A detector device can be used to determine intraocular pressure. For example, a detector device can be configured to provide a light source and a wavelength detector. The light source can be configured to direct a beam of light onto a patient's eye such that light is reflected from an implanted device containing a flexible filter membrane having one or more pressure sensors. The wavelength detector can then detect the wavelength of the reflected light. This detector can be a spectral measuring instrument capable of measuring the diffracted wavelength. As described herein, the measured wavelength can be correlated to the amount of flexing within the flexible filter and used to determine the intraocular pressure that resulted in that amount of flexing. In some cases, the detector device can record the wavelength measurements, the intraocular pressure values converted from the wavelength measurements, or both. In addition, any recorded values can be associated with the particular time and day the measurements were obtained. For example, a patient can take three measurements a day for a month, and the detector device can record the time, day and intraocular pressure value for each of those measurements. Determining intraocular pressure can allow patients and clinicians to determine whether or not the implanted device is plugged or clogged with, for example, debris such as polypeptides. In addition, close, real time monitoring of intraocular pressure can be used to assess the condition of a patient's glaucoma. In some cases, a detector device can be configured to transmit intraocular pressure measurements, for example, from a patient's home to the patient's doctor's office.

Solutions containing particles coated with protein/polypeptide dissolving/unplugging material (e.g., proteases, surfactants, and/or heparin) can be used to remove or reduce the amount of protein/polypeptide debris that may accumulated within an implanted device. For example, a clogged implanted device can be unclogged by administering an eye drop composition containing particles so coated. In general, the proteases can cleave polypeptides within the implanted device, thereby reducing the amount of polypeptide debris. Surfactants can block hydrophobic interactions, thereby preventing protein/polypeptide plugging/adherence. The particles can be micro/nano particles. For example, the particles can be 1 to 100 nm in diameter. Compositions containing such coated particles (e.g., protease-coated particles) can be any type of composition including, without limitation, eye drop solutions. In some cases, the particles can be biodegradable. For example, the particles can be designed to degrade within 1 or more (e.g., 2, 3, 4, 5, or more) hours after being applied to a human eye. Examples of biodegradable materials that can be used to make biodegradable particles include, without limitation, thermoplastic starch materials, mater-bi, polylatic acid, and poly-hydroxybutyrate-co-hydroxyvalerate. Any type of protein/polypeptide dissolving or unplugging material can be coated onto a particle including, without limitation, papain, subtilisin, or other proteases, or a surfactant (e.g., Triton X-100), or heparin.

In general, a composition containing particles coated with protein/polypeptide dissolving or unplugging material can be applied topically to the eye. In some cases, the material of the coating can be in an entirely liquid form without including any particles. In either case, the solution can have access to the external filter directly. In addition, once applied, the solution can diffuse into the anterior chamber. Once in the anterior chamber, the solution, with or without particles, can leave the eye through the filter membrane of the device. In some cases, a charged biopolymer can be applied to the filter membrane of the device, and the particles can be made to have an opposite charge, thereby allowing the particles to be attracted to the pores.

With reference to FIG. 1A, body 3 of the device is preferably formed of a material selected from the group consisting of silicone, acrylic, polyimide, polypropylene, polymethyl methacrylate, polydimethylsiloxane, and expanded polytetrafluoroethylene (preferably denucleated and coated with laminin). These materials are well known in the art and methods of fabricating tubular structures from such materials also are well known. The material from which the device is fabricated can be selected to provide bulk biocompatibility, as described above. The bulk properties of the material can be selected to impart rigidity as close as possible to that of the surrounding tissue, e.g. sclera.

A device provided herein can be of sufficient length to provide fluid communication between the anterior chamber 2 and tear film 4 when the device is implanted in the sclera 6 of an afflicted eye. In general, to provide fluid communication between the anterior chamber and tear film, the devices provided herein can have a minimum length of about 2 mm. In preferred embodiments, a device can have a length of at least about 2.5 mm. In general, a device can have a length of between about 2.5 mm and about 5 mm. The preferred length of at least about 2.5 mm can reduce the possibility of blockage of the lumenal opening in the anterior chamber by the iris. The length of the device within the scleral tract can be greater than the scleral thickness because insertion may not be perpendicular to the sclera, but rather more tangential to be parallel to the iris.

As shown in FIG. 1, the body 3 of the device can define a generally tubular lumen 5. In preferred embodiments, the lumen can have a diameter less than or equal to about 0.5 mm. On its external surface 10, the body 3 can preferably include a porous cellular ingrowth coating 15 on at least a portion thereof. Preferably, and as shown in FIG. 1A, the portion of the external surface coated with the cellular ingrowth coating 15 can correspond substantially to the portion of the body in contact with eye tissue (i.e., sclera) following scleral implantation. Such porous cellular ingrowth coatings have been described with respect to other ophthalmic implants, and have been made of silicone with a reported thickness of 0.04 mm. Selected growth factors can be adsorbed on to this coating to enhance cellular ingrowth.

Other surfaces of the device such as the entire lumenal surface 12, the portion of the external surface 10 not in contact with the sclera, and the inflow (14) and outflow (16) faces of the filter can further include coatings to enhance surface biocompatibility. Such coatings can include bio-inert polymer coatings such as phosphoryl choline (PC), polyethylene glycol (PEG), hydroxyethylmethacrylate (HEMAPC), poly[2 hydroxyethylmethacrylate] (PHEMA), and polyethylene oxide (PEO), and such bio-inert surface coatings may be further modified with biologically active molecules such as heparin, spermine, surfactants, proteases or other enzymes, or other biocompatible chemicals amendable to surface immobilization. The PEG concentration can be very high (e.g., in the range of 10 mol percent). Also, the PEG can be applied by plasma deposition, which can allow coating of the pore sidewalls.

Both PC and PEO polymer coatings can downregulate deleterious biological reactions, primarily by attracting a large and stable hydration shell when grafted onto a surface. PEO also can be amendable to end-group coupling for surface immobilization of biologically active molecules, which might include heparin, spermine, surfactants, proteases (e.g., papain) or other enzymes or chemicals. The addition of such bioactive molecules could advantageously impart specific desired functionality, for example, allowing a further increase in the hydrophilicity of the surface. Hydrophobic surfaced microporous filters are known to be much more prone to protein plugging than are microporous filters with hydrophilic surfaces.

Alternatively, instead of applying bio-inert surface coatings, all or parts of the device can be fabricated from a highly biocompatible polymer. Such a polymer can be fabricated by mixing a substrate polymer with a bio-inert polymer, such as PEG. This can lessen the need for surface coatings, or can make the bond between the substrate and the surface coating very strong because each can contain the same bio-inert polymer.

In the portion of the external surface of the body 3 that is in contact with eye tissue following implantation, the body can include a barb or barbs 17 designed to engage with tissue upon implantation and provide stability to the implanted device. The barb or barbs 17 can be formed as part of the device body during manufacture or can be fused or bonded to the device body by suitable means known in the art. The device can also be beveled at its first end 7 to aid in the implantation process.

The devices provided herein can include a filter capable of providing outflow resistance to aqueous humor flowing through the lumen of the device from the anterior chamber into the tear film. The filters employed in a device provided herein preferably are microporous/nanoporous filter membranes.

In FIG. 1, a microporous filter membrane 11 is shown at the second end 9 of the body 3. The microporous filter membrane 11 can include inflow face 14, outflow face 16, and can be circumscribed by peripheral edge 18. The size of the pores in the filter-membrane 11 at the exterior surface of the device preferably are approximately 0.2μ, or smaller. This can be sufficiently small enough to prevent ingress of all known bacteria. It can also be about the same pore size as has been shown to be present in the capsule formed around Molteno implant plates, and through which aqueous humor flows by simple, passive diffusion. That capsule is known to act as an “open sieve” for passage of latex microspheres of 0.2μ and smaller. The filter-membrane of this device would be expected to act as such an “open sieve,” but with a predetermined resistance to outflow to result in a low to normal intraocular pressure. The design parameters of microporous membranes suitable for use in a device provided herein can be summarized as follows.

Porous media theory can allow for the calculation of the resistance of a fluid through a porous structure by using the formula: resistance=8×fluid viscosity×length of pore/number of pores×π×pore radius to the fourth power. The viscosity of aqueous humor is essentially the same as saline, and the viscosity is stable. The pore radius could vary only over a range that would still permit it to act as a barrier to bacteria. The length of the pores, however, may be varied, and is determined by the thickness of the filter-membrane. The number of pores can also be varied to arrive at a desired resistance. Even though the eye's natural outflow is compromised in glaucoma, it is rarely zero, and would in most cases allow for a certain tolerance in the system even after a device provided herein is in place. In fact, the main natural outflow of the eye, the conventional or trabecular meshwork pathway, can be intraocular pressure dependent. The trabecular meshwork pathway can serve as a one-way valve, so when the intraocular pressure is very low, the trabecular meshwork is compressed with very little outflow, or backflow, allowed through it. When the intraocular pressure increases, to a certain level, the outflow can increase also.

In some embodiments, it is desirable to achieve a normal aqueous humor outflow resistance of about 3.2 mmHg×min/μL. In some embodiments, it is desirable to achieve an outflow resistance that produces a low normal intraocular pressure. For example, if a filter membrane with a diameter of 1.0 mm is used, that would result in a filter membrane area of 785,000 square μ. If a pore density of 40% of the filter membrane surface area is used, there would be ten 0.2μ pores/square μ. Thus, there would be a total of 7,850,000 pores of 0.2μ size. Using a filter membrane thickness of 100μ, the porous membrane theory equation for resistance would be:

$$\begin{matrix}R = 8 \times viscosity \times pore length \text{/} pore number \times \\ π \times pore radius to the fourth power \\ = 8 \times 1 \times 100 / 7 , 850 , 000 \times 3.14 \times .00001 \\ = 800 / 247 \\ = 3.2 ,\end{matrix}$$

the mean value for outflow resistance of normal, non-glaucomatous, eye.

Because episcleral venous pressure would not be a factor in the function of this device, as it is in the determination of normal intraocular pressure [e.g., P(ocular)=F(inflow)/C(facility of outflow)+P(evp)], the IOP with this device might be expected to be below normal. Alternatively, the outflow through the device, rather than the outflow resistance, could be adjusted to give the desired intraocular pressure.

Microporous filter membranes that have been used with ophthalmic devices or research include Nuclepore polycarbonate filter membranes, millipore filters, and microperforated silicone membranes. However, filter-membrane nanotechnology, and specifically microelectromechanical systems (MEMS)-based technology, can be useful to fabricate microporous membranes, in accordance with this document, to be optimally biocompatible, non-degradable, and immunoisolating. Substrates for nanofabrication of the devices provided herein can include, without limitation, silicon, metals, or polymers such as silastic, rubber, and gel materials. Examples of such technologies that are known and characterized in the art include:

(1) Microfabricated silicon(e) or silicon(e)-based biocapsules, an example of which would be polycrystalline silicon filter-membranes micromachined to present a high density of uniform pores, as small as 0.02μ.

(2) Microporous polymer networks, an example of which would be a polyurethane network formed by cross-linking a mixture of linoleic acid and a linear poly (etherurethane) with dicumyl peroxide. Microporosity is introduced by adding salt crystals before cross-linking and leaching it out afterwards. Pore size in this instance is 0.3-0.7μ, with a membrane thickness of 8μ. But, both pore size and membrane thickness can be varied.

(3) Fiber networks with a porous structure, an example of which would be an acrylonitrile membrane (AN 69).

(4) Microcapsules based on the use of oligomers which participate in polyelectrolyte complexion reactions.

The application of these technologies to medicine has heretofore been most prominently related to pancreas cell transplantation.

In FIG. 1, the microporous filter membrane 11 can be attached at its periphery 18 to the body 3 at the second end 9 of the body. The lumenal opening at the second end can thus be closed by the microporous filter membrane. As shown in FIG. 1A, and in preferred embodiments of this document, the filter 11 can be bonded, fused or otherwise attached to the body at the second end of the device, most preferably at the edge of the second end defining the lumenal opening, such that the filter is substantially flush with the second end of the body. Although preferred, such placement of the filter is not required. The filter can be placed elsewhere, for example, in a slightly recessed or protruding position, or at any position along the lumen of the body. In some embodiments, the filter can be formed of the material used to fabricate the device body and be integral with it. In such cases, manufacture of the device could occur as a one-step fabrication process to fabricate the tubular body which would be closed at one end (corresponding to the second end of the ultimate device) with body material of a desired thickness. A microporous filter membrane can then be fabricated at the closed end by creating a desired number of pores of appropriate diameter, by perforation or other suitable means. This device could then be implanted in the sclera as described herein.

As shown in FIG. 1A, the fixation of the filter membrane, by fusion, bonding, or other means of attachment, can result in a one-piece device that can be implanted as such in the sclera of an afflicted eye. The shape of the filter membrane can preferably be either round or oval. In some embodiments, filters such as microporous/nanoporous filter membranes, debris filters, or flexible filters can be connected to the body of the device via heat shrinking. For example, a device containing a flexible, microporous/nanoporous filter membrane can be made by heat shrinking a flexible, microporous/nanoporous filter membrane to the body of the device. In such cases, the body and/or the flexible filter can be made of a heat shrinkable material. Examples of heat shrinkable materials include, without limitation, polyolefin, polypropylene, polytetrafluoroethylene, polyvinylchloride, and polyester.

As also shown in FIG. 1, the body 3 of the device can flare at the second end 9, and the filter and second end 9 of the device can be situated substantially flush with the external scleral surface 21. The flaring of the body at its second end 9 can aid in the flush mounting of the device in the eye by providing an endpoint of insertion as the device is pushed into the sclera during surgery. The device 1 can also be beveled at its first end 7 to assist in implantation. In this embodiment, the diameter of the filter membrane can thus exceed the diameter of the lumen in the portion of the body that is not flared. The degree to which the body flares and the resultant diameter of the microporous filter membrane may be adjusted to optimize the functional properties of the filter membrane. With the second end of the device, including the filter, in communication with the tear film, the filter can be readily accessible for cleaning, using methods involving vacuum, chemical, enzymatic, micro backflushing, magnetic pulsing, or ultrasonic disruptive processes.

FIG. 1B depicts a device, as shown in FIG. 1A, implanted in an eye with like numbers signifying like features. The view shown is an external view of an eye showing the external, intrascleral, and intra-anterior chamber portions of the device shown in FIG. 1A implanted in the eye. A frontal view of the second end 9 and filter 11 (with outflow face 16 and peripheral edge 18 visible) is shown, and the device can extend through the sclera 6 and into the anterior chamber 2. The flaring of the second end 9 of the device within the sclera is shown, and the second end can be substantially flush with the scleral surface.

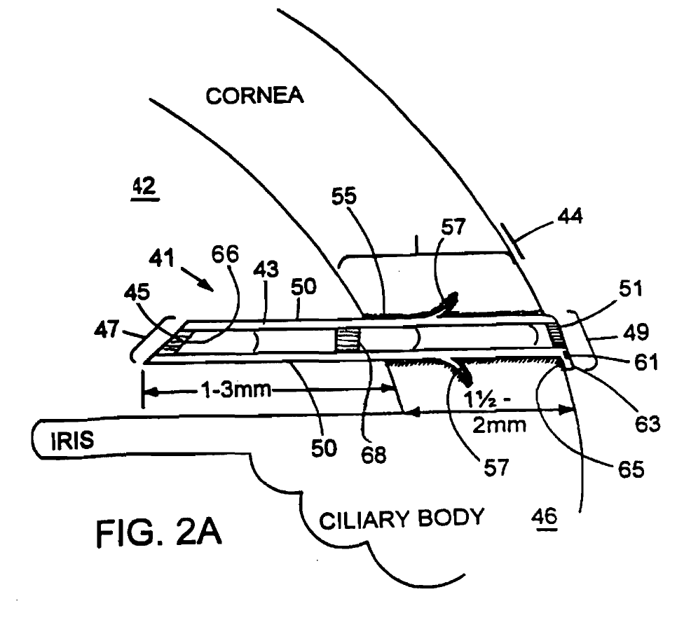

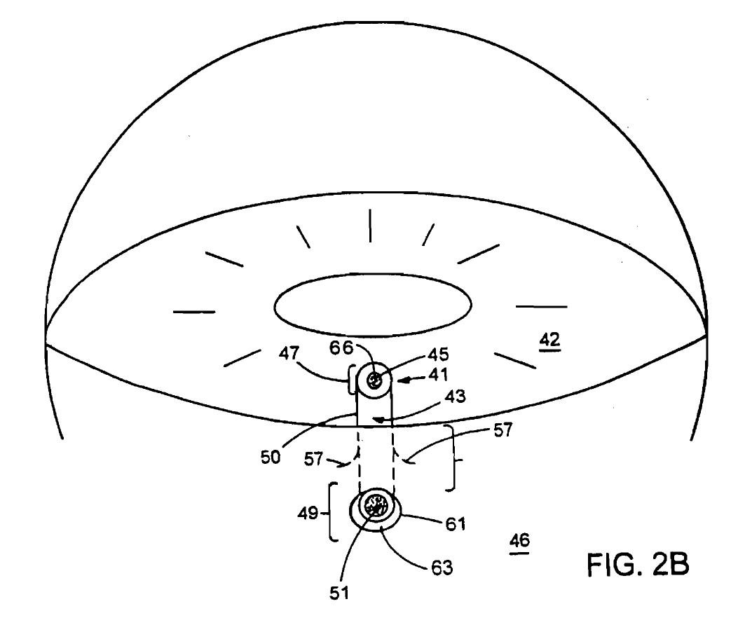

FIGS. 2A and 2B show another embodiment of a device provided herein, with like numbers signifying like features. The views of the device embodiment shown in FIGS. 2A and 2B are similar to those shown in FIGS. 1A and 1B. The features of the devices shown in FIGS. 1A/1B and 2A/2B are similar in all respects except where noted. A device 41 is shown, having a body 43, a lumen 45, a first end 47, and a second end 49. Also shown are filter 51, porous cellular ingrowth coating 55, stabilization barbs 57, and a bevel at the first end 47. As with other embodiments, the device 41 can be of sufficient length to allow fluid communication between the anterior chamber 42 and tear film 44 of an eye through the lumen 45 when implanted in the sclera 46.

In the embodiment shown in FIGS. 2A and 2B, the device can comprise a head portion 61 which is not substantially flush with, but rather extends externally to the scleral surface. The body 43 of the device can be adapted to form a lip 63 at the second end 49 of the device. The lip 63 can extend around at least a portion of the filter 51 of the device (shown as extending for roughly ¾ of the circumference of the head portion 61). The lip 63 can have an external lip surface 65 that is continuous with the external surface 50 of the body. The lip 63 can serve to stabilize the device against the scleral surface, and the external lip surface 65 can be provided with porous cellular ingrowth coating 55 (as shown in FIG. 2A) to further stabilize the device in the eye. The lip 63 can further provide an endpoint of insertion when the device is implanted.

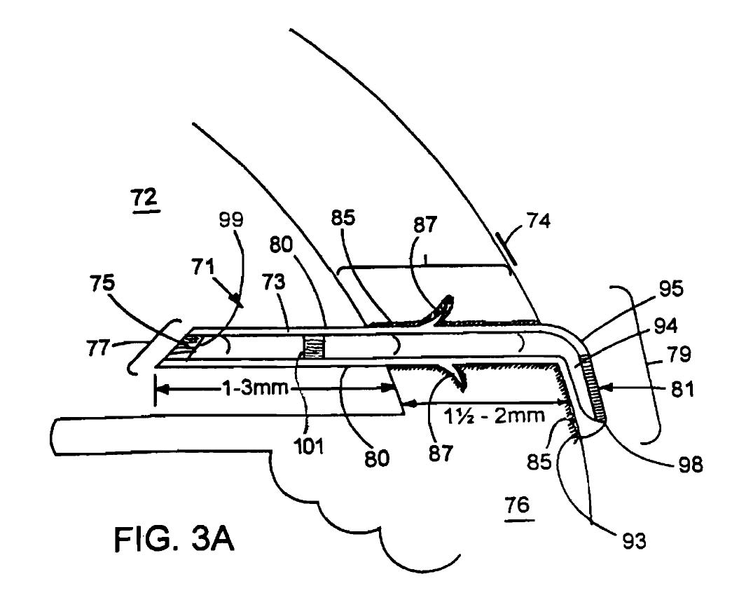

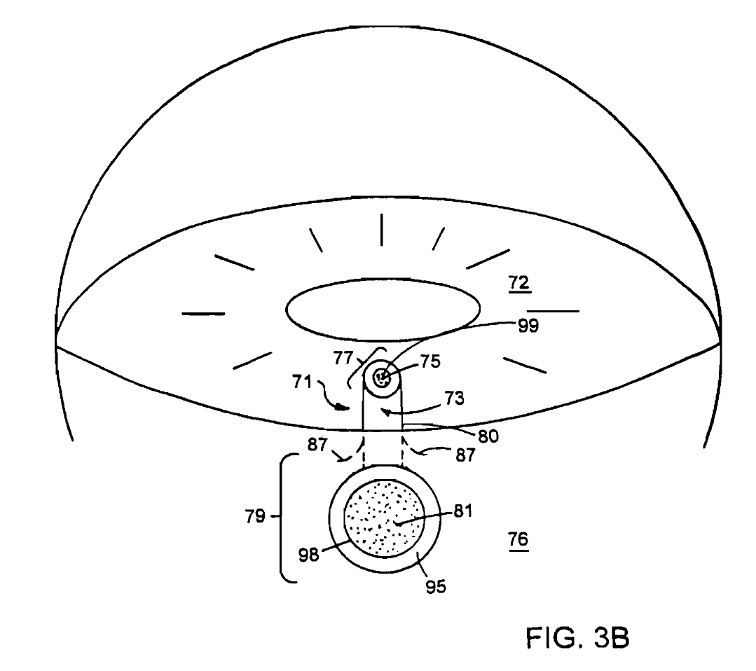

FIGS. 3A and 3B depict still another embodiment illustrative of a device provided herein, with like numbers signifying like features. The view of the device embodiment shown in FIGS. 3A and 3B are similar to those shown in FIGS. 1A and 1B. The features of the devices shown in FIGS. 1A/1B and 3A/3B are similar in all respects except where noted. A device 71 is shown, having a body 73, a lumen 75, a first end 77, and a second end 79. Also shown are filter 81, porous cellular ingrowth coating 85, stabilization barbs 87, and a bevel at the first end 77. The device can be of sufficient length to allow fluid communication between the anterior chamber 72 and the tear film 74 when the device is implanted in the sclera 76.

In the embodiment shown in FIGS. 3A and 3B, the device can comprise, at its second end 79, a disc-shaped head portion which is not flush with, but rather extends externally to the scleral surface. The body 73 of the device can be adapted to form the disc portion, which includes a cavity 94 (FIG. 3A), which can be in communication with the lumen 75. The disc-shaped head portion can have opposing inner and outer faces 93 and 95, respectively. The inner face 93 (continuous with the external surface 80 of the body) can be in contact with the external surface of the sclera 76, and the outer face 95 as shown in FIG. 3A includes the filter 81. The inner face 93 can be coated with porous cellular ingrowth coating 85. In preferred embodiments, a peripheral edge 98 of the filter 81 can be contiguous with the periphery of the body 73 at the opening to the cavity 94, such that the filter 81 forms part of the outer face 95 of the disc-shaped head portion.

In another embodiment, a device provided herein can include an additional debris filter, or debris filters, within the lumen of the body, to keep debris from the filter membrane that is fabricated to provide the desired outflow resistance. Preferably, a debris filter can be positioned at or near the first end 7 of the body of the device, within the anterior chamber of the eye. The debris filter can contain larger pores than the resistance-providing microporous filter membrane, for example in the range of 1μ in diameter. While any porous filter will necessarily provide some resistance to flow through it, the debris filter(s) can be fabricated to provide the least possible resistance. The primary function of the debris filter can be to keep debris from reaching the microporous filter membrane, which is the outflow resistance determining element. Porous media flow theory teaches that resistance is inversely proportional to the pore radius to the fourth power, so a much larger pored filter would provide little resistance to aqueous humor outflow. Number and length of pores can also be varied to eliminate most resistance.

While the microporous filter membrane of the device that provides outflow resistance would have modifications, especially related to its surface chemistry, to prevent adherence of proteins or cells, limiting its exposure to potentially plugging debris may also be important. An additional debris filter can be placed at or near the first end of the device body to block most blood and pigment cells and cell fragments that might be included in the aqueous humor outflow. The surface of the debris filter preferably is accessible for laser photodisruption of accumulated debris, as is used to eliminate debris that occasionally collects on the surface of intraocular lens. Because this additional filter can preferably be covering the inner, beveled, end of the lumen, its surface area can be increased, and it can be facing anteriorly. The larger surface area can allow for some plugging before any significant resistance develops to outflow; and an anterior orientation can make laser access easier.

In addition to placing such a filter at the inner end of the body of the device, a similar debris-collecting filter can be positioned at or near the second end 9 of the body, with the resistance-providing filter membrane internal to it at some position within the lumen.

Referring to the figures, a flexible filter is shown as 28 in FIG. 1a, as 68 in FIG. 2a, and as 101 in FIG. 3a. Referring to the figures, a debris filter is shown as 26 in FIGS. 1a and 1b, as 66 in FIGS. 2a and 2b, and 99 in FIGS. 3a and 3b. The debris filter can be flexible as described herein. For example, a debris filter can be designed to flex in response to changes in intraocular pressure, thereby altering outflow resistance.

The additional, larger pored debris filter(s), designed to keep debris from the filter membrane, can be fabricated using various micromachining techniques, including microelectromechanical systems (MEMS)-based technology, as with the filter membrane. Alternatively, soft lithography or focused ion beam (FIB) technologies may be employed. Laser perforations could also be used to create the pores. Potential materials for fabrication of the debris filter include silicon or silicone, polytetrafluoroethylene, polypropylene, polymethyl methacrylate, acrylic, polyurethane, polyimide, hydrogels, and other polymers, whether flexible or not.

As with the filter membrane, the debris filter(s) can be preferably bonded to the body within the lumen. The bond can provide a robust, permanent, and totally hermetic seal. Examples of suitable bonding methodologies are fusion, wafer, covalent, or anodic bonding; or the use of various biocompatible adhesives, including silicone elastomer, epoxy, cyanoacrylate, or polyurethane; or a heat shrinking process.

As with the rest of the device exposed to aqueous humor, the debris filter(s) preferably has surface modifications to make it as bioinert as possible. Surface coating using self-assembled monolayers of biomolecules may be used; examples include phosphoryl choline, polyethylene oxide, or polyethylene glycol. These can provide a very hydrophilic surface, thereby decreasing/eliminating protein and cellular adhesion.

The method for installing this device is simple and consumes little time. Sometime before installation, topical antibiotic and non-steroidal anti-inflammatory drops (NSAID) can be applied to the operative eye. These can be continued for one week postoperatively four times a day. The NSAID can help stabilize the blood-aqueous barrier.

All embodiments of the device illustrated herein may be inserted under topical anesthesia, possibly supplemented subconjunctivally. In general, the devices provided herein can be inserted into the sclera using routine operative procedures. The location of insertion for all embodiments can be in the sclera at about the posterior surgical limbus. The device could be inserted at any site around the limbus, but would preferably be inserted at the far temporal limbus.

The insertion procedure is begun by excising a small amount of conjunctiva at the site of the anticipated insertion, exposing the underlying sclera. Any bleeding can then be cauterized. For embodiments of the device as shown in FIG. 2 and FIG. 3, a superficial layer of sclera may be excised beneath the anticipated position of the exterior portion of the device. This can allow these embodiments to be more flush with the surrounding external scleral surface, as occurs easily with the embodiment of FIG. 1.

Then, approximately 1-2 mm posterior to the limbus, at the site of the now exposed sclera, a diamond blade can be used to make a stab incision into the anterior chamber, while held roughly parallel to the iris. This blade can be of a size predetermined to make an opening into the anterior chamber sized appropriately for the introduction of the device. This stab incision can be made gently, but relatively quickly, assiduously avoiding any and all intraocular structures. Such an uneventful paracentesis has been found not to disrupt the blood-aqueous barrier in most cases. In any event, any disruption of this barrier is usually of less than 24 hours duration without continued insult. In the embodiment of the device shown in FIG. 1, the paracentesis could be customized to the flared external shape of the device by using a diamond blade, or trochar, sized to the device, and fitted with a depth guard. This can insure accurate and predictable depth of insertion so the exterior surface of the device would lie flush with the external scleral surface.

The device is next picked up and held with a non-toothed forceps. The lips of the stab incision wound may be gaped with a fine, toothed forceps. The pointed tip of the tube element would then be gently pushed through the scleral tract of the stab incision and into the anterior chamber, with the tube lying above and parallel to the iris, with the bevel up [i.e., anteriorly]. Alternately, a dedicated instrument could be used to facilitate placement of the device. This instrument can consist of a hollow tube within which the device could be placed, and guided into the paracentesis wound. The instrument can have a mechanism to extrude the device into its proper position. The flare in the embodiment of FIG. 1, the external lip in the embodiment of FIG. 2, and the disc portion in the embodiment of FIG. 3 can provide for a definite endpoint to the depth of insertion. For the embodiments of the device having a beveled first end, the bevel can be oriented anteriorly so as to minimize the potential for blockage of the lumenal opening by the iris. The scleral barb(s) then can stabilize the device until the biointegration with the sclera is complete. This biointegration can be a function of its porous cellular ingrowth surface, likely enhanced by adsorbed growth factors. In the embodiment of FIG. 3, a 10-0 nylon suture on a broad spatula needle may be used to suture the disc portion into the sclera, providing additional stability to the device until the biointegration is complete. This suture may then be easily removed. In the embodiments of FIGS. 1 and 2, a suture could also be used to add additional temporary stability.

After insertion of the device, an ocular shield should be placed over the eye.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device for treating glaucoma in an eye, comprising:
a body defining a lumen and having first and second ends and external and lumenal surfaces, said body having a length sufficient to provide fluid communication between the anterior chamber and tear film of an eye through said lumen when said device is implanted in the sclera; and
a flexible filter membrane capable of providing outflow resistance to aqueous humor flowing through said lumen and capable of flexing in response to an increase in intraocular pressure.

2. The device of claim 1, wherein said second end of said device is adapted to lie substantially flush with the scleral surface when said device is implanted in the sclera.

3. The device of claim 1, wherein said body is flared at said second end.

4. The device of claim 1, wherein said body comprises a material selected from the group consisting of silicone, acrylic, polyimide, polypropylene, polymethyl methacrylate, polytetrafluoroethylene, hydrogels, polyolefin, polyvinylchloride, and polyester.

5. The device of claim 1, wherein said flexible filter membrane comprises polydimethylsiloxane, a silicone rubber, or a hydrogel.

6. The device of claim 1, wherein said flexible filter membrane is a microporous/nanoporous filter membrane or a debris filter.

7. The device of claim 1, wherein said flexible filter membrane is a microporous/nanoporous filter membrane and comprises micropores having a diameter less than or equal to about 0.2 microns.

8. The device of claim 1, wherein said flexible filter membrane is a debris filter and comprises pores having a diameter between about 0.5 and 2 microns.

9. The device of claim 8, wherein said debris filter comprises an inflow face, an outflow face, and a peripheral edge contiguous with said body.

10. The device of claim 1, wherein said device comprises a microporous/nanoporous filter membrane and a debris filter.

11. The device of claim 10, wherein said debris filter is positioned at said first end or between said first end and said microporous/nanoporous filter membrane.

12. The device of claim 11, wherein said flexible filter membrane is positioned between said debris filter and said microporous/nanoporous filter membrane.

13. The device of claim 10, wherein said microporous/nanoporous filter membrane comprises a pressure sensor.

14. The device of claim 13, wherein said pressure sensor comprises photonic crystals.

15. The device of claim 14, wherein said photonic crystals are within a polymer network of a hydrogel.

16. The device of claim 10, wherein said body and said microporous/nanoporous filter membrane comprise the same material.

17. The device of claim 16, wherein said body and said microporous/nanoporous filter membrane are fused together using heat.

18. The device of claim 17, wherein said body and said microporous/nanoporous filter membrane comprise polyolefin, polypropylene, polytetrafluoroethylene, polyvinylchloride, or polyester.

19. The device of claim 10, wherein said device comprises a second debris filter.

20. The device of claim 19, wherein said second debris filter is positioned at or near the second end of the body, external to said microporous/nanoporous filter membrane.

21. The device of claim 1, wherein the flexing of said flexible filter membrane in response to an increase in intraocular pressure reduces said outflow resistance.

22. The device of claim 1, wherein said flexible filter membrane comprises a pressure sensor.

23. The device of claim 22, wherein said pressure sensor comprises photonic crystals.

24. The device of claim 23, wherein said photonic crystals are within a polymer network of a hydrogel.

25. The device of claim 1, wherein said body and said flexible filter membrane comprise different materials.

26. The device of claim 25, wherein said body and said flexible filter membrane are fused together using heat.

27. The device of claim 25, wherein said body and said flexible filter membrane comprise polyolefin, polypropylene, polytetrafluoroethylene, polyvinylchloride, or polyester.

28. A method for treating glaucoma, comprising:
(a) providing a device comprising a body defining a lumen and having first and second ends, said body having sufficient length to provide fluid communication between the anterior chamber and tear film of an eye, and said device comprising a flexible filter membrane capable of providing outflow resistance to aqueous humor and capable of flexing in response to an increase in intraocular pressure; and
(b) implanting said device in the sclera of the eye such that aqueous humor flows from the anterior chamber to the tear film of the eye.

29. A method for making a device for treating glaucoma in an eye, said method comprising using heat to fuse a body to a filter membrane to form said device, wherein said body comprises a lumen, first and second ends, and external and lumenal surfaces, said body having a length sufficient to provide fluid communication between the anterior chamber and tear film of an eye through said lumen when said device is implanted in the sclera, and wherein said filter membrane is capable of providing outflow resistance to aqueous humor flowing through said lumen.

30. The method of claim 29, wherein said body and said filter membrane comprise different materials.

31. The method of claim 30, wherein said body comprises a heat shrink material.

32. The method of claim 30, wherein said material is selected from the group consisting of polyolefin, polypropylene, polytetrafluoroethylene, polyvinylchloride, and polyester.

33. A method for reducing polypeptide clogging in a device implanted in the sclera of an eye, said method comprising administering a solution comprising particles containing a protease, a surfactant, heparin, or a combination thereof to said eye under conditions wherein polypeptides clogging said device are cleaved or removed.

34. The method of claim 33, wherein said device comprises a body defining a lumen and having first and second ends, said body having sufficient length to provide fluid communication between the anterior chamber and tear film of the eye, and said device comprising a filter membrane capable of providing outflow resistance to aqueous humor.

35. The method of claim 34, wherein said device comprises a flexible filter membrane capable of flexing in response to an increase in intraocular pressure.

36. The method of claim 35, wherein said flexible filter membrane is said filter membrane.

37. The method of claim 33, wherein said solution is a biocompatible solution.

38. The method of claim 33, wherein said solution is an eye drop solution.

39. The method of claim 33, wherein said particles are capable of degrading following administration to said eye.

40. The method of claim 33, wherein said particles comprise material selected from the group consisting of thermoplastic starch materials, mater-bi, polylatic acid, and poly-hydroxybutyrate-co-hydroxyvalerate.

41. The method of claim 33, wherein said protease is a papain or subtilisin protease.

42. A method for providing a patient with the ability to monitor intraocular pressure, comprising:
(a) providing a patient with a detector comprising a light source and a wavelength sensor, wherein the sclera of an eye of said patient comprises (i) a device comprising a body defining a lumen and having first and second ends and external and lumenal surfaces, said body having a length sufficient to provide fluid communication between the anterior chamber and tear film of said eye through said lumen and (ii) a flexible filter membrane capable of providing outflow resistance to aqueous humor flowing through said lumen and capable of flexing in response to an increase in intraocular pressure, wherein said flexible filter membrane comprises a pressure sensor; and
(b) instructing said patient to emit light from said detector onto said eye such that said detector is capable of detecting the wavelength of the emitted light that is reflected from said pressure sensor.

43. The method of claim 42, wherein said pressure sensor comprises photonic crystals.

44. The method of claim 43, wherein said photonic crystals are within a polymer network of a hydrogel of said flexible filter membrane.

45. The method of claim 42, wherein said light is emitted as white light.

46. The method of claim 42, wherein said detector records the wavelength of the emitted light that is reflected from said pressure sensor.

47. The method of claim 42, wherein said detector converts the detected wavelength of the emitted light that is reflected from said pressure sensor into a pressure value.

48. The method of claim 42, wherein said detector records the wavelength value of the emitted light that is reflected from said pressure sensor or a pressure value converted from said wavelength value, wherein said recorded wavelength value or pressure value is recorded with the time, day, or time and day that said detector detected said wavelength.

49. The method of claim 42, said detector records multiple wavelength values detected by said detector at different times or multiple pressure values converted from said multiple wavelength values.

50. A method for determining intraocular pressure in a patient, wherein the sclera of an eye of said patient comprises (i) a device comprising a body defining a lumen and having first and second ends and external and lumenal surfaces, said body having a length sufficient to provide fluid communication between the anterior chamber and tear film of said eye through said lumen and (ii) a flexible filter membrane capable of providing outflow resistance to aqueous humor flowing through said lumen and capable of flexing in response to an increase in intraocular pressure, wherein said flexible filter membrane comprises a pressure sensor, wherein said method comprises:
(a) providing a detector comprising a light source and a wavelength sensor; and
(b) emitting light from said detector onto the eye of said patient such that said detector is capable of detecting the wavelength of the emitted light that is reflected from said pressure sensor.

51. The method of claim 50, wherein said pressure sensor comprises photonic crystals.

52. The method of claim 50, wherein said photonic crystals are within a polymer network of a hydrogel of said flexible filter membrane.

53. The method of claim 50, wherein said light is emitted as white light.

54. The method of claim 50, wherein said detector records the wavelength of the emitted light that is reflected from said pressure sensor.

55. The method of claim 50, wherein said detector converts the detected wavelength of the emitted light that is reflected from said pressure sensor into a pressure value.

56. The method of claim 50, wherein said detector records the wavelength value of the emitted light that is reflected from said pressure sensor or a pressure value converted from said wavelength value, wherein said recorded wavelength value or pressure value is recorded with the time, day, or time and day that said detector detected said wavelength.

57. The method of claim 50, said detector records multiple wavelength values detected by said detector at different times or multiple pressure values converted from said multiple wavelength values.

58. A kit comprising a device and a detector, wherein said device comprises (a) a body defining a lumen and having first and second ends and external and lumenal surfaces, said body having a length sufficient to provide fluid communication between the anterior chamber and tear film of an eye through said lumen when said device is implanted in the sclera, and (b) a flexible filter membrane capable of providing outflow resistance to aqueous humor flowing through said lumen and capable of flexing in response to an increase in intraocular pressure, wherein said flexible filter membrane comprises a pressure sensor; and wherein said detector comprises a light source and a wavelength sensor, wherein said detector is capable of emitting light onto an eye containing said device such that said detector is capable of detecting the wavelength of the emitted light that is reflected from said pressure sensor.
